(19) United States
(12) Patent Application Publication
(10) Pub. No.: US 2013/0201446 A1
(43) Pub. Date: Aug. 8, 2013

(54) Hologram

(71) Applicants: Simon Richard Geoffrey Hall, London Greater London (GB); Richard Frederick Stevens, London Greater London (GB)

(72) Inventors: Simon Richard Geoffrey Hall, London Greater London (GB); Richard Frederick Stevens, London Greater London (GB)

(21) Appl. No.: 13/698,459

(22) PCT Filed: May 19, 2011
(86) PCT No.: PCT/GB2011/050952

§ 371 Date: Jan. 28, 2013

(30) Foreign Application Priority Data
May 19, 2010 (GB) 1008338.4

(51) Int. Cl. A61B 3/032; A61B 3/00; A61H 5/00; G03H 1/04; G01M 11/02; G03H 1/02; A61B 3/08

(52) U.S. Cl. CPC A61B 3/032; G03H 1/0272; A61B 3/0091; A61B 3/08; G03H 1/041; G01M 11/0207; A61H 5/00
USPC 351/201; 359/22; 351/239; 351/205; 359/30; 356/124; 601/37

(57) ABSTRACT

A substrate includes a diffracting structure providing a hologram which encodes one or more holographic images. Every image of the one or more holographic images has a holographic location at either a finite or substantially infinite effective optical distance. The hologram is to be used as an ophthalmic eye chart or an eye exercise device or to provide a scale for measuring distances.

The present invention relates to a hologram, as well as to apparatus and a method of generating a hologram. The preferred embodiment generates at least one virtual image located at a prescribed distance from the viewer's eyes or at infinity which for ophthalmic applications can be approximated by a distance of at least 6 to 8 metres from the viewing position. The viewer's accommodation and focus adjust naturally to the image at the apparent distance reconstructed by the hologram (or, for eye diagnostics, to infinity).

In many instances it is desirable or necessary to allow a person to view an image which appears to the person at an infinite distance, typically at least 6 or at least 8 metres from the person. Viewing an image at such a distance is useful for ophthalmic purposes as well as for eye exercise, to cite just two examples.

For instance, an optician's chart should be located from the viewer by a distance of at least 8 metres and this is generally achieved using a mirror to fold the path to make it more compact.

U.S. Pat. No. 7,384,144 utilizes laser speckle and conventional optics to produce an infinity image. This fixation target is preceded by Scientifica Cook Ltd device Laserspec which uses a rotating diffusing drum illuminated by a laser to provide an infinity fixation speckle target for the human eye. This is described in: Letter to the Editor, Physics World 1 Oct. 1988 Peter Kalmus, Queen Mary College “Test Your Own Eyesight”.

U.S. Pat. No. 5,040,888 discloses a hologram system which helps reduce eye strain.

Ophthalmic examining rooms are often not large enough to produce a real 8 m optical path to the sight chart. Other methods use bulky expensive equipment with limited portability, for instance as disclosed in U.S. Pat. No. 7,384,144. Binocular devices cause problems as there is the risk of eye or other infections from contact with the eyepieces, also spectacle frames cannot be worn.

The present invention seeks to provide a hologram, and apparatus and method of generating a hologram.

According to an aspect of the present invention, there is provided a substrate including a diffracting structure providing a hologram, said hologram encoding one or more holographic images, every image of the one or more holographic images having a holographic location at a substantially infinite effective optical distance from the substrate.

When such a diffracting structure is illuminated appropriately with reference light, a virtual object appears. A virtual object created by a hologram in this way is referred to in this description and the accompanying claims as an image or holographic image. The virtual object does not generally appear to be located at the substrate, but at a distance from the substrate. The position at which the virtual object appears to be located is referred to in this description and the accompanying claims as its holographic location.

Preferably, a substantially infinite effective optical distance is an effective optical distance of at least 6 metres, preferably of at least 8 metres.

A preferred embodiment, provides an easy to use eye exercise device. By having every holographic image at a substantially infinite optical distance, a user can easily glance at the device for a moment and immediately see a holographic image at an effectively infinite optical distance from him, allowing him to exercise his eyes briefly before returning to his previous task.

In an embodiment of the invention, an eye chart includes a substrate as described above, wherein the one or more holographic images are a plurality of holographic images of different predetermined sizes. Preferably, the images are consistent in size with those of an conventional optician's chart, being progressively smaller in graded steps This can remove the need for a long optical path around an optician's office and can enable the eye chart to be located close to the patient.

This can enable a patient to be located on one side of the eye chart, viewing the holographic images, and an optical observation or measurement device to be located on the opposite side of the eye chart, the device being operable to observe or take measurements of eyes of the patient. In some embodiments, this can include observing eye behaviour, or measuring the distance between a patient's pupils while the patient's eyes are focused at infinity.

According to an aspect of the invention, there is provided a measurement device including: a substrate including a diffracting structure providing a hologram, said hologram encoding at least one holographic image at a predetermined effective optical distance from the substrate, the at least one holographic image providing a calibration scale representing distance.

The calibration scale may include a plurality of elements of the at least one holographic image for example marking predetermined intervals along a distance axis.

The holographic image could provide image elements which depict respective units of a scale.

This can be utilised for example in a microscope in which a holographic image, when brought into focus, is able to overlay an image of an object under study with distance indicators appropriate for that level of magnification to enable the user to measure the object under study.

Preferably, the at least one holographic image is a plurality of holographic images, each image of the plurality of holographic images having a holographic location at a predetermined effective optical distance from the substrate, the predetermined effective optical distance from the substrate of each image of the plurality of holographic images being different from the predetermined effective optical distances from the substrate of the other images of the plurality of holographic images;

wherein the plurality of holographic images provides a scale of distance from the substrate.

According to an aspect of the invention, there is provided a method of calibrating an optical device capable of variable focus, including:

providing a measurement device as described above;

aligning the optical device with the substrate so that an effective optical distance of an image of the plurality of holographic images from the substrate is substantially equal to an effective optical distance of that image from an imaging plane of the optical device;

illuminating the substrate with reference light to cause the plurality of holographic images to appear, and operating the optical device to successively focus different images of the plurality of holographic images on the imaging plane; calibrating the optical device by associating a configuration of the optical device, when a particular image of the plurality of holographic images is focused on the imaging plane, with known characteristics of that image.

According to an aspect of the invention, there is provided a method of manufacturing a hologram, including:

illuminating an object with a first light beam so that light scattered from the object passes through an optical element, such as a convex lens, to a light sensitive medium, wherein the optical element modifies the scattered light to be as if scattered from an object at a substantially infinite optical distance from the light sensitive medium;

illuminating the light sensitive medium with a second light beam which is coherent with the first light beam; and subsequently

manufacturing a hologram derived from the light sensitive medium.

Such a method is able to facilitate preparation of a holographic image the holographic location of which is at a substantially infinite optical distance from the hologram within the confines of a small holography bench.

According to an aspect of the present invention, there is provided a substrate including a hologram bearing an image at an effective distance of at least 6 metres from the substrate. Advantageously, the image is at an optical distance representing infinity.

In an embodiment, the hologram includes a plurality of images, said images having different predetermined effective optical distances. The images are preferably at regular and set optical distances from one another, that is along the line of sight. For instance, there may be one or more images at an effective infinite distance and one or more images at effective closer distances, such as at metre intervals or other predetermined distances. Having a plurality of images at predetermined effective optical distances can be useful in determining optical characteristics as well as in measurement applications.

According to another aspect of the present invention, there is provided an optometric eye chart including a plurality of holographic images. In an embodiment, the images have different sizes. Preferably, the images have predetermined effective optical distances. In an embodiment the images have effective optical distances of at least 6 metres, preferably of at least 8 metres. In an embodiment, the images may have different optical distances, such as at metre or shorter intervals. Having images at different effective optical distances can diagnose different eye conditions such as myopia and hyperopia.

Embodiments of the invention provide a stereoscopic acuity test device including a substrate including a diffracting structure providing a hologram, said hologram encoding a plurality of holographic images having holographic locations at different predetermined effective optical distances from the substrate.

Objects represented by images of the plurality of holographic images having holographic locations at different predetermined effective optical distances from the substrate can be different sizes such that the images representing the objects appear to be the same size.

The plurality of holographic images can include images at least three different effective optical distances from the substrate.

According to another aspect of the present invention, there is provided a method of producing a holographic image including the step of providing an object at an effective distance of at least 6 metres from a holographic medium and recording an image of the object on the medium. Advantageously, the method provides a plurality of objects at different predetermined distances from the holographic medium and records images of each of the objects on the medium.

The predetermined distances are preferably spaced at set and advantageously also at regular distances from one another.

The preferred embodiment provides a hologram of an object or objects optically located at known distances from the holographic media. This, when illuminated correctly, produces a wavefront matching that of the original objects, producing images at the same set distances from the hologram.

The preferred embodiments taught herein enable the space requirement for a fixation image device to be simply dictated by the illumination system for the hologram. The hologram can be compact, lightweight, easily replicated and relatively inexpensive compared to devices with multiple optical components. A bright image can be produced which will attract attention even in a brightly lit environment.

The hologram reconstructs the wavefront from the distant object without the alignment and eyestrain problems sometimes associated with stereoscopic and binocular devices.

Embodiments of the present invention are described below, by way of example only, with reference to the accompanying drawings, in which:

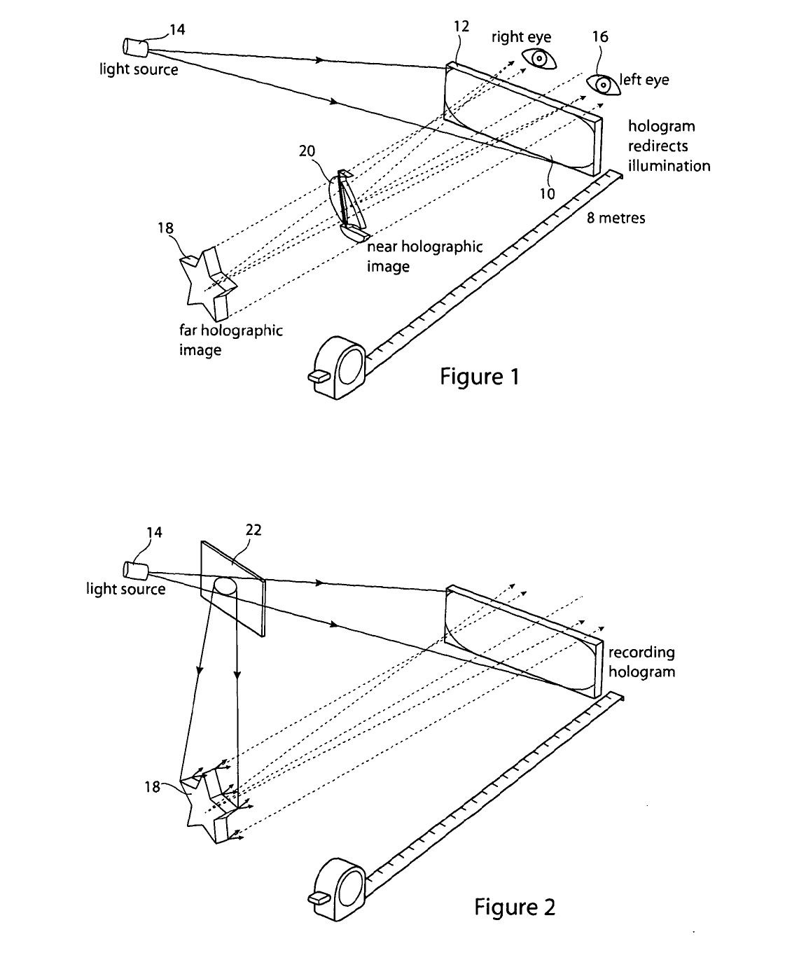

FIG. 1 is a schematic diagram of the preferred embodiment of infinity image hologram in use;

FIG. 2 is a schematic diagram showing the apparatus and method for the recordal of a holographic image at a specific distance;

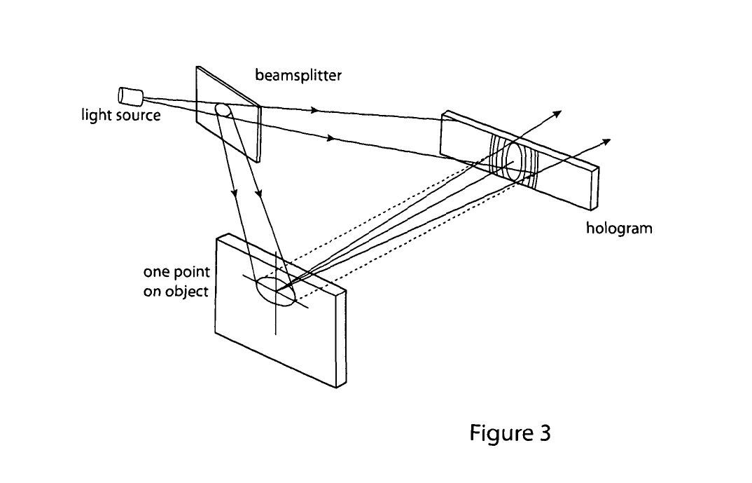

FIG. 3 is a schematic diagram of a method of recording a hologram of one object point;

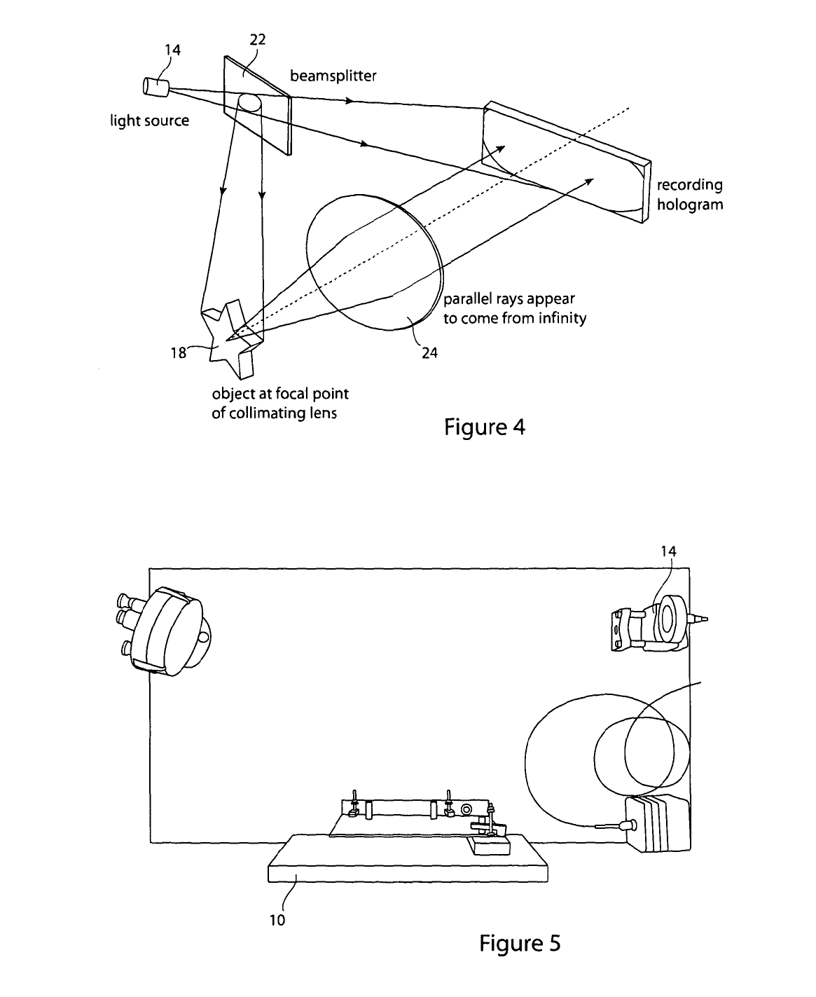

FIG. 4 is a schematic diagram showing the apparatus and method for the recordal of an infinity holographic image;

FIG. 5 is a plan view of an embodiment of hologram recordal apparatus;

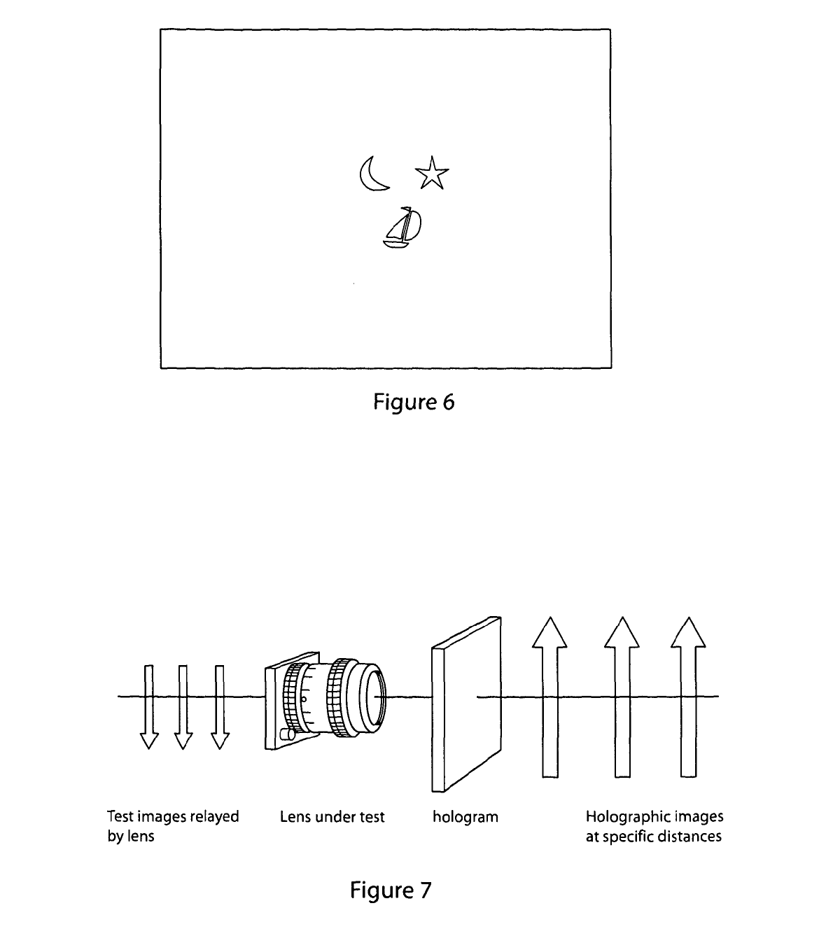

FIG. 6 is an example of a simple infinity image produced by the apparatus and method;

FIG. 7 is a schematic diagram of a method of using holographic images at specific distances to calibrate depth scales on optical systems;

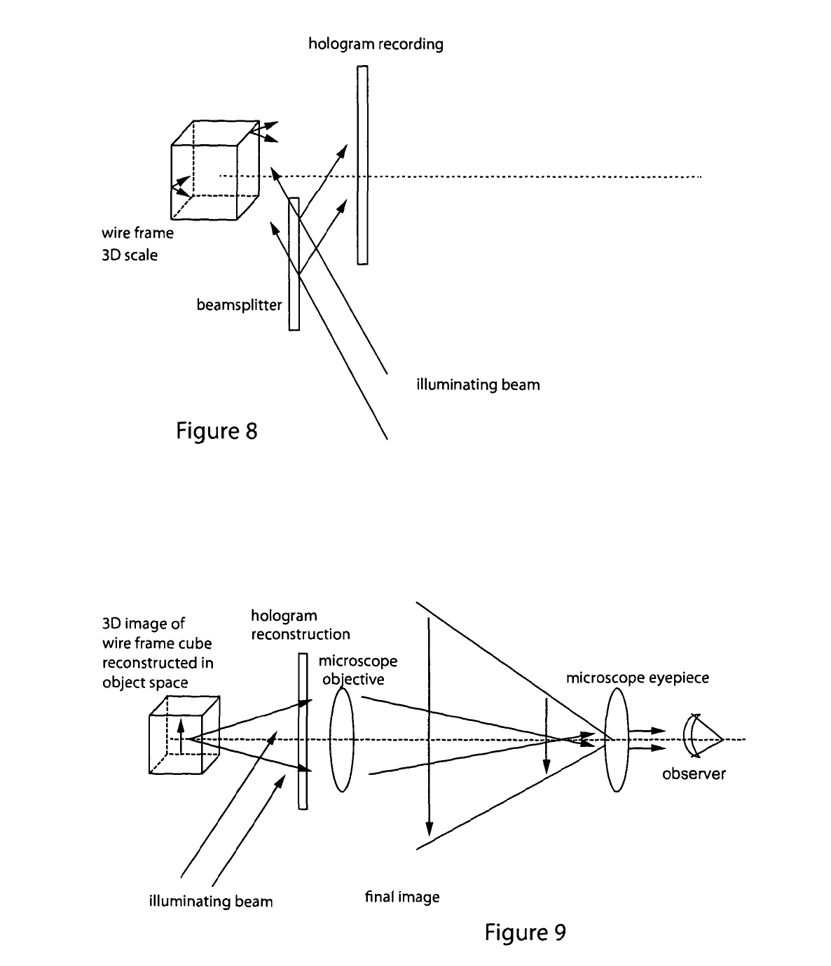

FIG. 8 is a schematic diagram of a method of recording a hologram of a 3D wire frame; and

FIG. 9 is a schematic diagram of a method of using the hologram recorded by the method of FIG. 8 in a microscope field.

In one embodiment of the invention, shown in FIG. 1, a diffracting structure (the hologram) 10 formed on a thin substrate 12 would redirect light from a point source 14 towards the observer 16 and reconstruct a wavefront that is similar to that from an object 18, 20 at a set and measured distance from the holographic media 10. At least one of the objects 18 can be at infinity for ophthalmic purposes. In another embodiment, an object 18, 20 can be viewed through an optical system to give the appearance of being at a calculated distance (or infinity) from the holographic media 10.

Referring to FIG. 2, the hologram 10 is fabricated by recording in a light sensitive medium the interference pattern generated by two coherent wavefronts, one arising from the object 18 and the other from a reference source formed of light source 14 and a beam splitter 22. The wavefront from the object should travel 8 metres (at least) or should travel through an optical component such as a convex lens 24 as shown in FIG. 4, which modifies the wavefront from the object 18 so that it is equivalent to a wavefront produced by an object at a greater distance than could be easily arranged on a holography bench (or beyond 8 metres for an ophthalmology target) from the recording medium 10.

The recording is processed to yield a diffracting structure that when illuminated under specific conditions redirects light to an observer who sees an image at infinity. Any residual light that is not diffracted in the required direction passes through the hologram 10 and can be dumped.

A holographic image may be reconstructed by transmission through a hologram or reflection from a hologram.

Reflection holograms usually work by reflection and diffraction at successive layers (Bragg layers) in the depth of the recording medium. An advantage of the reflection hologram is that the reconstruction only occurs when the wavelength and angle of incidence of the illumination beam match the optical characteristics of the hologram defined by the angle and spacing of the Bragg diffracting layers.

As described above, a hologram is made by illuminating an object with coherent light such as that from a laser or a spectrally filtered source. The object to be recorded is illuminated and light scattered by the object is directed to fall on a layer of photosensitive material. Some of the light from the source is made to fall directly on the photosensitive layer, bypassing the object and acting as a reference wavefront. The scattered light and reference wavefronts combine optically to produce an interference pattern of light and dark bands which are recorded in the photosensitive layer. The recording is processed to yield a structure that when illuminated with a replica of the reference wavefront will diffract light and form a replica of the object wavefront.

Examples of light-sensitive media for recording holograms include photographic emulsion (very fine grain), photopolymers, photoresists, photothermoplastics, and dichromated gelatin

Photographic emulsion records the variation in intensity in the holographic interference pattern. Where light falls on the silver halide crystals energy is absorbed and silver atoms are released to form a latent “image”. The latent image is developed to form a permanent silver image. In a further process the silver image is converted to a transparent form in which the original variations in light intensity are represented by variations in refractive index in the hologram. This improves the light diffraction efficiency of the hologram compared to the original amplitude recording.

Other recording media can be processed directly to produce diffracting structures that operate on the phase of the illuminating wavefront by introducing optical path variations due to either variations in thickness of the recording medium or variations in refractive index of the recording medium.

A hologram records and reconstructs both the intensity and phase in a wavefront that appears to come from an object. It records the information in the form of an interference pattern, generated by combining the wavefront from the object and a separate reference wavefront.

FIG. 3 shows how a hologram of a single point on an object is recorded. The interference pattern between wavefronts emanating from two points is a series of concentric rings of equal intensities.

It is this pattern that is recorded and reproduced as optical path variations in the hologram. The hologram is reilluminated with a copy of the reference wavefront and light is diffracted to form a wavefront that appears to originate from the position of the object point. The same mechanism holds for recording and reconstructing all the other points on the object enabling a 3D image to be reconstructed.

The fine pattern of amplitude variations in the recording is processed to form a transparent structure or diffraction grating, in which the optical path lengths correspond to the phase variations in the interference pattern. When illuminated with a replica of the original reference wavefront, light is diffracted to form a replica of the original wavefront from the object. When this wavefront is viewed by the human eye or a camera an image of the original object is perceived. By moving the eye, parallax effects can be observed and by focusing the three dimensional nature of the image can be explored.

To recreate an image as seen when viewing the original object it is generally necessary to illuminate with a reference source in the same position as that used when recording. Depending on the process used to make the hologram it may be necessary to adjust the source position during reconstruction to obtain optimum brightness in the image. It may also necessary to adjust the source position to obtain optimum focus in the reconstructed image. If there is a change of wavelength between recording and reconstruction it may be necessary to adjust the source position for optimum results.

FIG. 5 is a plan view of an example of apparatus for generating an infinity holographic image.

FIG. 6 shows a plurality of simple holographic images which are located at different effective optical distances.

The hologram produced in the manner described above can be used in many different applications. One simple practical application is in the provision of a hologram which can be used for eye exercise, for instance in an office environment to provide the opportunity of staff to exercise their eyes at regular time intervals when working at close quarters. For instance, the hologram could be positioned close to a computer monitor, allowing a user to look at images at a distance (infinity) by looking at the hologram. This application can provide eyestrain relief for operators using visual display units or microscopes, for instance. It can be particularly effective when the or every image of the hologram is at infinity.

Another application provides an optometric eye chart, in the form of a hologram provided with a series of images, in one embodiment all at infinity (typically 6 to 8 metres and more) and of different sizes and in other embodiments images at different effective optical distances and of the same or different sizes. Thus in its simplest form, the hologram could replace conventional eye charts and mirror arrangements. In other forms, the hologram can test eyesight at different optical distances, for which it would be advantageous to have a series of images at different set distances and at set distal spacings from one another. This application can provide ophthalmic fixation target to enable the use of diagnostic and therapeutic instruments.

Other applications include advertising, for instance to provide an eye-catching advertising hologram with an image at infinity. This would lend itself well to certain products (for instance those incorporating landscape or parallax with holograms recorded at a shorter distance).

Another application can test for stereoscopic acuity using holographic images at different depths.

Another application, a depth gauge allows for the viewer to measure or estimate the distance to real objects by determining which of the holographic images coincides with the object under test using depth cues experienced by the viewer. It can also be used to calibrate distance measurement optical systems such as focusing scales on camera lenses using a hologram with either real or virtual images at known distances, as shown in FIG. 7.

As can be seen in FIG. 7, an optical device capable of variable focus, such as a camera, can be aligned with a hologram which encodes a plurality of holographic images at known effective optical distances, and can be successively focused on each of the holographic images. Since the holographic images are at known effective optical distances, the focal length of the camera can be determined, and the camera can be calibrated.

The hologram can be adapted for fitting to eyeglasses, to ensure that lenses are centered in the visual axis. This can be used in conjunction with a pupil spacing measurement system for better assurance in fitting spectacle frames for progressive lenses, and other specialty lenses, since small errors cause eye-strain in certain prescriptions.

The apparatus and method described herein can provide a simple lightweight optical device that achieves the effect of generating an image at infinity without the need for a heavy, complicated and bulky optical system. The hologram may be replicated at low cost for mass production. In use the hologram may be readily interchanged to give a different image. This means that infinity accommodation of human eyes can be achieved in a small space (without an 8 m optical path).

Another application is in the field of microscopy. A common requirement is to provide a scale to allow measurement of microscopic objects. A calibrated reticule often satisfies this requirement in one or two dimensions. Additionally, calibrated microscopic artefacts are used to provide a direct comparison. In embodiments of the invention, a hologram of the calibrated artefact can be used in the optical path to overlay a real or virtual 3D image of the artefact with the microscopic object. The example shown in FIGS. 8 and 9 is one implementation of this, but the hologram can be inserted at other points in the microscope optical path. This would involve the use of an optical system in between the hologram plate and the object used to make the hologram to compensate for the optical components and position in the microscope system. The size of the artefact recorded by the hologram can be different to the size of the image reconstructed by the hologram if relay optics are used to control the magnification between the object and the image recorded by the hologram.

The disclosures in United Kingdom patent application no. 1008338.4, from which this application claims priority, and in the abstract accompanying this applications are incorporated herein by reference.

1. A substrate including a diffracting structure providing a hologram, said hologram encoding one or more holographic images, every image of the one or more holographic images having a holographic location at a substantially infinite effective optical distance from the substrate.

2. An eye chart including a substrate according to claim 1, wherein the one or more holographic images are a plurality of holographic images of different predetermined sizes.

3. An eye chart according to claim 2, wherein the one or more holographic images is a series of at least three holographic images, the size of the images progressively increasing through the series by a predetermined ratio.

4. An eye chart according to claim 2, wherein the one or more images when produced are visible by looking towards the front of the substrate from a viewing position in front of the substrate and appear to be located to the rear of the substrate, the eye chart further including:
an optical observation or measurement device located to the rear of the substrate, the device being operable to observe or take measurements of eyes of a viewer at the viewing position.

5. An eye chart according to claim 4, wherein the device includes diagnostic equipment for use in diagnosing an eye condition.

6. An eye chart according to claim 4, wherein the substrate includes an optical path therethrough through which the device can observe or take measurements of eyes of a viewer at the viewing position.

7. An eye chart according to claim 6, wherein the optical path includes a path of transparent material through the substrate.

8. An eye chart according to claim 2, further including a second substrate, the second substrate providing a stereoscopic acuity test device, the second substrate including a second diffracting structure providing a second hologram, the second hologram encoding a second plurality of holographic images having holographic locations at different predetermined effective optical distances from the second substrate.

9. An eye chart according to claim 8, wherein objects represented by images of the second plurality of holographic images having holographic locations at different predetermined effective optical distances from the second substrate are different sizes such that the images of the objects appear to be the same size.

10. An eye chart according to claim 8, wherein the second plurality of holographic images includes images at least three different effective optical distances from the second substrate.

11. An eye exercise device including a display provided with a substrate, the substrate including a diffracting structure providing a hologram, said hologram encoding one or more holographic images, every image of the one or more holographic images having a holographic location at a substantially infinite effective optical distance from the substrate.

12. An eye exercise device according to claim 11, wherein every image of the one or more holographic images has a holographic location at an effective optical distance of at least 6 metres from the substrate.

13. An eye exercise device according to claim 10, wherein the one or more holographic images is a plurality of holographic images.

14. A measurement device including:
a substrate including a diffracting structure providing a hologram, said hologram encoding at least one holographic image at a predetermined effective optical distance from the substrate, the at least one holographic image providing a calibration scale representing distance.

15. A measurement device according to claim 14, wherein:
the at least one holographic image is a plurality of holographic images, each image of the plurality of holographic images having a holographic location at a predetermined effective optical distance from the substrate, the predetermined effective optical distance from the substrate of each image of the plurality of holographic images being different from the predetermined effective optical distances from the substrate of the other images of the plurality of holographic images;
wherein the plurality of holographic images provides a scale of distance from the substrate.

16. A measurement device according to claim 15, wherein the plurality of holographic images is a series of images marking regular intervals of effective optical distance from the substrate.

17. A measurement device according to claim 15, wherein each of the images of the plurality of holographic images includes a symbolic representation of its respective effective optical distance from the substrate.

18. A measurement device according to claim 15, further including an operator guide providing details of the predetermined effective optical distance of each of the plurality of holographic images.

19. A measurement device according to claim 15, further including an optical focusing device aligned with the substrate so that an effective optical distance of an image of the plurality of holographic images from the substrate is substantially equal to an effective optical distance of that image from an imaging plane of the focusing device, the focusing device having a variable focus lens operable to focus on the imaging plane an image of an object being measured and an image of the plurality of holographic images to determine the distance from the substrate at which an object is located.

20. A microscope including a measurement device, wherein the measurement device includes:
a substrate including a diffracting structure providing a hologram, said hologram encoding at least one holographic image at a predetermined effective optical distance from the substrate, the at least one holographic image providing a calibration scale representing distance;
wherein the microscope is operable to be optically focused on an object under study and the at least one holographic image so that the at least one holographic image appears to overlay the image of the object under study with a measurement scale.

21. A method of calibrating an optical device capable of variable focus, including:
providing a measurement device, wherein the measurement device includes a substrate including a diffracting structure providing a hologram, said hologram encoding at least one holographic image at a predetermined effective optical distance from the substrate, the at least one holographic image providing a calibration scale representing distance, wherein the at least one holographic image is a plurality of holographic images, each image of the plurality of holographic images having a holographic location at a predetermined effective optical distance from the substrate, the predetermined effective optical distance from the substrate of each image of the plurality of holographic images being different from the predetermined effective optical distances from the substrate of the other images of the plurality of holographic images, wherein the plurality of holographic images provides a scale of distance from the substrate;
aligning the optical device with the substrate so that an effective optical distance of an image of the plurality of holographic images from the substrate is substantially equal to an effective optical distance of that image from an imaging plane of the optical device;
illuminating the substrate with reference light to cause the plurality of holographic images to appear, and operating the optical device to successively focus different images of the plurality of holographic images on the imaging plane;
calibrating the optical device by associating a configuration of the optical device, when a particular image of the plurality of holographic images is focused on the imaging plane, with known characteristics of that image.

22. A method according to claim 21, wherein associating a configuration of the optical device, when a particular image of the plurality of holographic images is focused on the imaging plane, with known characteristics of that image includes associating a focal length of the configuration of the optical device with the predetermined effective optical distance of the particular image from the substrate.

23. A method according to claim 21, wherein associating a configuration of the optical device, when a particular image of the plurality of holographic images is focused on the imaging plane, with known characteristics of that image includes determining a dimensional scale for images produced in that configuration of the optical device by comparing a dimension of the particular image as measured with the aid of the optical device with a corresponding known dimension of a real-world object represented in the particular image.

24. A method according to claim 21, wherein the plurality of holographic images represent real-world objects of different sizes.

25. A method according to claim 21, wherein the optical device is selected from a group including a camera, a telescope, binoculars, a periscope and a microscope.

26. A method of manufacturing a hologram, including:
illuminating an object with a first light beam so that light scattered from the object passes through an optical element to a light sensitive medium, wherein the optical element modifies the scattered light to be as if scattered from an object at a substantially infinite optical distance from the light sensitive medium;
illuminating the light sensitive medium with a second light beam which is coherent with the first light beam; and subsequently
manufacturing a hologram derived from the light sensitive medium.

27. A method according to claim 26, wherein the optical element is a convex lens.
